(19) United States
(12) Patent Application Publication
(10) Pub. No.: US 2009/0306125 A1
(43) Pub. Date: Dec. 10, 2009

(54) Mitochondria-Targeted Antioxidant Prodrugs and Methods of Use

(76) Inventors: Marion W. Anders, Pittsford, NY (US); James L. Robotham, Rochester, NY (US); Shey-Shing Sheu, Pittsford, NY (US)

Correspondence Address: LICATA & TYRRELL P.C., 66 E. MAIN STREET, MARLTON, NJ, 08053, US

(21) Appl. No.: 12/094,618

(22) PCT Filed: Nov. 20, 2006
(86) PCT No.: PCT/US06/61081

§ 371 Date: Sep. 11, 2008

Related U.S. Application Data
(60) Provisional application No. 60/739,033, filed on Nov. 22, 2005.

(51) Int. Cl. A61K 31/4375; C07D 471/04; A61K 31/437; C07D 233/84; A61K 31/4172; C12N 5/06

(52) U.S. Cl. 514/300; 546/122; 546/113; 548/324.1; 514/398; 435/375

(57) ABSTRACT

The present invention is a mitochondria-targeted antioxidant prodrug useful for the prevention or treatment of diseases or conditions associated with mitochondrial dysfunction resulting from changes in the mitochondrial redox environment. Antioxidant prodrugs of the invention are produced by modifying an antioxidant to a fatty acid so that the resulting prodrug is targeted to and activated by an enzyme of mitochondrial fatty acid beta-oxidation.

### BACKGROUND OF THE INVENTION

Physiologically, mitochondria perform a variety of key cellular regulatory processes, including ATP production, intracellular Ca²⁺ regulation, reactive oxygen species (ROS) generation and detoxication, and apoptosis (Tzagoloff (1982) Mitochondria, Plenum Press, New York). Mitochondria use approximately 90% of the consumed O₂ for oxidative phosphorylation and ATP synthesis. Thus, the proteins involved in the mitochondrial electron transport chain are probable sites for ROS generation. Intracellular glutathione, glutathione peroxidase, glutathione transferases, catalase, superoxide dismutase, and a variety of other antioxidant defenses keep ROS concentrations in check, which allows cells to function homeostatically thereby preventing oxidative stress (Abid, et al. (2004) J. Biol. Chem. 279:44030-44038; Zhang, et al. (2002) J. Virol. 76:355-363; Li, et al. (2000) Cancer Res. 60:3927-3939; Warner, et al. (1996) Am. J. Physiol. 271:L150-L158; Schiavone & Hassan (1988) J. Biol. Chem. 263:4269-4273). A shift in the balance between ROS generation and destruction to overproduction or decreased detoxication is associated with chronic diseases (Ross, et al. (1997) Am. J. Kidney Dis. 30:489-494).

The etiology of a range of diseases is associated with the generation of excess reactive oxygen species. Steady-state maintenance of ROS/antioxidant ratio is, however, essential for cell signaling. Reactive oxygen species generated in cells include the superoxide anion radical (O₂.⁻), hydrogen peroxide (H₂O₂), hypochlorous acid (HOCl), hydroxyl radical (OH.), and singlet oxygen (¹O₂). These ROS are formed as a consequence of endogenous enzymatic and nonenzymatic reactions within the cell and within mitochondria. ROS may also be formed in response to external stimuli and chemicals.

ROS are generated by normal biochemical reactions in the cell. Leakage of electrons from the mitochondrial electron transport chain is a significant source of mitochondrial ROS, particularly superoxide (Boveris & Cadenas (1997) In: Oxygen, Gene Expression, and Cellular Function, Clerch & Massaro, eds., Marcel Dekker, New York, pp. 1-25). Moreover, the TCA cycle enzymes α-ketoglutarate dehydrogenase and the pyruvate dehydrogenase complex also generate superoxide and H₂O₂ (Starkov, et al. (2004) J. Neurosci. 24:7779-7788). Superoxide is also generated by NADPH oxidase, which is found in phagocytic and nonphagocytic macrophages (Quinn & Gauss (2004) J. Leukoc. Biol. 76:760-781), and by xanthine dehydrogenase/oxidase (Rajagopalan (1997) In: Biotransformation, Guengerich, ed., Elsevier, N.Y. pp. 165-178). Hydrogen peroxide is produced by mitochondrial monoamine oxidase (Cashman (1997) In: Biotransformation supra pp. 69-96) and by the superoxide dismutase (MnSOD and Cu/ZnSOD)-catalyzed dismutation of superoxide (Fridovich (1995) Annu. Rev. Biochem. 64:97-112). In addition, peroxisomal acyl-CoA oxidases also generate hydrogen peroxide (Reubsaet, et al. (1988) Biochim. Biophys. Acta 958:434-442). The myeloperoxidase-catalyzed generation of hypochlorous acid is an important line of defense against invading microorganisms (Winterbourn, et al. (2000) Curr. Opin. Hematol. 7:53-58).

There is no known enzymatic route to detoxify the hydroxyl radical, which may be produced by the Haber-Weiss reaction in the presence of transition metals, particularly iron. Singlet oxygen may be formed by photodynamic processes or from the reaction of hypochlorous acid with hydrogen peroxide.

Reactive nitrogen species have also been implicated in cell damage and death. Nitric oxide synthase catalyzes the synthesis of the radical species nitric oxide (NO.), which may react with superoxide to give peroxynitrite (ONOO⁻). ROS generation may also be associated with external stimuli. UV and high-energy irradiation, the metabolism of some xenobiotics, air pollutants (O₃), and the redox cycling of quinones and nitroaromatics are all associated with ROS generation.

The balance between these sources of ROS depends on the physiologic and pathophysiologic states of the organism, and it is often difficult to pinpoint the source of ROS generation. It is, however, known that ROS exert important regulatory functions (Droge (2002) Physiol. Rev. 82:47-95). Hence, a basal or tonal concentration of ROS, especially at the level of the mitochondrion, is essential for basic cell signaling processes. In other words, all ROS are not created equal, and compartmentalization and concentration gradients are highly important. Abolishment of all cellular ROS by vigorous use of antioxidants may not be beneficial and, indeed, may prove harmful. The requirement for a basal ROS tone may explain why many antioxidant-based therapies have failed.

Mitochondria are attractive targets for drug-delivery strategies because of their roles in cellular energy metabolism, programmed (apoptotic) cell death, calcium homeostasis, and cell signaling. Moreover, mutations in mitochondrial DNA are associated with a range of human diseases, again making mitochondria attractive targets for mitochondrial gene therapy. Hence, strategies have been developed to target small and large molecules with therapeutic potential to mitochondria (Muratovska, et al. (2001) Adv. Drug Deliv. Rev. 49:189-198; Weissig (2003) Crit. Rev. Ther. Drug Carrier Syst. 20:1-62; Weissig, et al. (2004) Drug Design Rev.-Online 1:15-28).

For example, the high potential gradient across the mitochondrial inner membrane can be exploited to deliver lipophilic cations to mitochondria. Cationic compounds, such as rhodamine 123 and tetraphenylphosphonium (TPP⁺), have been adopted for mitochondrial membrane potential determinations and a series of cationic antioxidants that preferentially accumulate in mitochondria have been developed (Ross, et al. (2005) Biochemistry (Moscow) 70:222-230). Further, a triphenylphosphonium-based, mitochondria-targeted mixture of ubiquinol (mitoquinol) and ubiquinone (mitoquinone), i.e., MitoQ (Kelso, et al. (2001) J. Biol. Chem. 276:4588-459), as well as MitoVit E (Smith, et al. (1999) Bur. J. Biochem. 263:709-716); MitoPBN (Murphy, et al. (2003) J. Biol. Chem. 278:48534-48545); MitoPeroxidase, a mitochondria-targeted analog of ebselen (Filipovska, (2005) J. Biol. Chem. 280:24113-24126); and glutathione choline ester (MitoGSH) and N-acetyl-L-cysteine choline ester (MitoNAC) have been synthesized for delivery of an antioxidant to mitochondria to selectively prevent mitochondrial oxidative damage.

### SUMMARY OF THE INVENTION

The present invention is a mitochondria-targeted antioxidant prodrug composed of a selected, fatty acid-modified antioxidant which is activated by an enzyme of mitochondrial fatty acid beta-oxidation. In particular embodiments, the antioxidant prodrug is in admixture with a pharmaceutically acceptable carrier to form a pharmaceutical composition.

The present invention is also a method for producing a mitochondria-targeted antioxidant prodrug by modifying a selected antioxidant to a fatty acid to produce a mitochondria-targeted antioxidant prodrug which is activated by an enzyme of mitochondrial fatty acid beta-oxidation.

Use of the mitochondria-targeted antioxidant prodrug in methods for decreasing mitochondrial dysfunction resulting from changes in the mitochondrial redox environment and preventing or treating a disease associated with mitochondrial dysfunction is also provided.

### DETAILED DESCRIPTION OF THE INVENTION

The present invention relates to antioxidant prodrugs which are specifically targeted to the mitochondria. The prodrug antioxidants of the instant invention can advantageously be used in the prevention and treatment of diseases associated with mitochondrial dysfunction resulting from changes in the mitochondrial redox environment because the instant prodrugs primarily exert their effects upon the mitochondria but may also exert their effects in other compartments of the cell.

As used in the context of the present invention, a prodrug is a compound that undergoes biotransformation via a metabolic process before exhibiting its pharmacological effects. Prodrugs are generally viewed as drugs containing specialized non-toxic protective groups used in a transient manner to alter or to eliminate undesirable properties in the parent molecule until the target site is reached. In accordance with the instant invention, an antioxidant prodrug is said to be targeted to the mitochondria by virtue of the unique mitochondrial localization of fatty acid β-oxidation enzymes that activate or release the antioxidant from its prodrug form within the mitochondria.

The term antioxidant, as used in the context of the instant invention, refers to a compound that, when present at low concentrations compared to those of an oxidizable substrate, significantly delays or prevents oxidation of that substrate. There is an abundance of oxidizable substrates in the cell, including proteins, lipids, carbohydrates, and DNA. Thus, antioxidants can function to prevent the formation of or to detoxify free radicals, to scavenge ROS (e.g., superoxide, hydrogen peroxide, hypochlorous acid, ozone, singlet oxygen, hydroxyl radical, and peroxyl, alkoxyl, and hydroperoxyl radicals) or their precursors.

Particular embodiments of the instant invention embrace a selected antioxidant compound, wherein a selected antioxidant is defined as an antioxidant containing a suitable phenolic, hydroxyl, or thiol group which can be modified to a fatty acid such that the modified antioxidant serves as a substrate of, and is activated by, an enzyme of fatty acid β-oxidation.

Suitable selected antioxidants with phenolic groups include chain-breaking phenol- and pyridinol-based antioxidants such as Vitamin E compounds including, for example, tocopherol (e.g., alpha-tocopherol, beta-tocopherol, gamma-tocopherol, delta-tocopherol), tocoquinone, tocotrienol (e.g., alpha-tocotrienol, beta-tocotrienol, gamma-tocotrienol, delta-tocotrienol), and analogues of Vitamin E such as TROLOX®, a compound which is more hydrosoluble than natural forms of Vitamin E; and synthetic antioxidants such as 2,6-dimethyl-4-methoxyphenols (see, e.g., U.S. Pat. No. 4,552,682) and 6-amino-3-pyridinols (see, e.g., Wijtmans, et al. (2003) Angewandte Chemie 115:4506-4509).

Selected antioxidants with suitable hydroxyl groups include, but are not limited to, hydroxylamines such as N-substituted hydroxylamines including N-alkylhydroxylamines (e.g., N-tert-butylhydroxylamine, N-methylhydroxylamine); benzylhydroxylamines; and the like. Other suitable antioxidants include analogs of α-phenyl-N-tert-butylnitrone that contain a hydroxyl group.

Exemplary selected antioxidants with available thiol groups include, but are not limited to, thiol-based 4-mercaptoimidazole antioxidants such as 1,5-dimethyl-4-mercaptoimidazole; dithiols such as 1,2-dithiol-3-thiones (e.g., 5-[p-methoxyphenyl]-3H-1,2-dithiol-3-thione, 4-methyl-5-pyrazinyl-3H-1,2-dithiole-3-thione), ethane-1,2-dithiol and propane-1,3-dithiol; and the like. See, e.g., 4-mercaptoimidazoles disclosed in U.S. Pat. No. 6,056,965 and Spaltenstein, et al. (1987) J. Org. Chem. 52:2977-79.

A fatty acid modification embraced by the present invention is an aryloxyalkanoic acid-based prodrug of a phenol- or pyridinol-based antioxidant, and chain lengthened analogs thereof. As used herein, an antioxidant prodrug based upon a particular fatty acid is intended to mean that the fatty acid is used to deliver the specified antioxidant to the mitochondria. For example, 3-arylpropanoic acids and 5-aryloxypentanoic acids, which after one cycle of beta-oxidation yield 3-arylpropanoic acids, can effectively be used to deliver an antioxidant. Encompassed within the scope of the present invention are aryloxyalkanoic acids with a variety of fatty acid chain lengths and heteroatom positions. Moreover, ester, amide, alcohol and other functional derivatives of an aryloxyalkanoid acid are contemplated. As exemplified herein, a chain lengthened 3-(2,2,5,7,8-pentamethylchroman-6-yl)propanoic acid, synthesized according to the general method disclosed in U.S. Pat. No. 6,770,672, can be employed wherein upon beta-oxidation 2,2,5,7,8-pentamethylchroman-6-ol is produced. Preparation of 3-aryloxypropanoic acid-based prodrugs of 4-mercaptoimidazole-based antioxidants can be carried out as exemplified herein by reacting a 4-mercaptoimidazole such as 1,5-dimethyl-4-mercaptoimidazole with an ethyl acrylate or ethyl 3-bromopropionate, which after hydrolysis, affords the 4-mercaptoimidazole-based antioxidant prodrug.

Alternatively, thiol-based antioxidants such as the dithiol antioxidants can be readily modified by cleaving dimethylacetal and reacting the resulting aldehyde with the diothiol antioxidant so that subsequent hydrolysis affords the desired dithiol-based prodrug.

Further embraced by the present invention is a 3-aminoxypropanoic acid-based prodrug of a hydroxylamine. As exemplified herein, a hydroxylamine antioxidant such as an N-substituted hydroxylamine can be modified to yield its 3-aminoxypropanoic acid-based prodrug by Michael addition of the N-substituted hydroxylamine to acrylamide and subsequent hydrolysis.

Antioxidants for use in preparing the prodrugs of the present invention can be isolated from a natural source or wholly or partially synthetically- or recombinantly-produced. Methods for isolating or producing antioxidants or antioxidant extracts are well-established in the art, see, e.g., U.S. Pat. Nos. 6,770,672; 6,737,552; 6,660,320; 6,656,358; 6,653,530; 6,623,743; RB38,009; 6,429,356; 6,436,362; 6,262,279; 6,410,290; 6,231,853; and 5,714,362 and WO 91/04315.

While specific antioxidants and the preparation of prodrugs thereof are disclosed herein, such disclosure in no way limits the types antioxidants that could be modified to a fatty acid to serve as a substrate of, and be activated by, a fatty acid β-oxidation enzyme.

Mitochondria-targeted prodrugs of the present invention are activated by the fatty acid β-oxidation enzymatic machinery present in mitochondria. Advantageously, xenobiotic fatty acids, such as N-substituted 3-aminoxypropanoic acids and 3-aryloxypropanoic acids are short- or medium-chain fatty acids that enter mitochondria directly without the necessity for acyl carnitine formation and are converted to their acyl-CoA thioesters by ligases present in mitochondria (Vessey, et al. (1999) Biochim. Biophys. Acta 1428:455-462). Moreover, there are several examples of the biotransformation and bioactivation of xenobiotic fatty acids by mitochondria. S-(1,2-Dichlorovinyl)-3-mercaptopropionic acid is directly toxic to mitochondria without prior acyl-CoA or carnitine ester formation (Stonard (1973) Biochem. Pharmacol. 22:1329-1335; Stonard & Parker (1971) Biochem. Pharmacol. 20:2417-2427). This potent mitochondrial poison, as well as 5,6-dichloro-4-thia-5-hexenoic acid and related 5,6,6-trihalo-4-thiahexanoic acids, undergo fatty acid β-oxidation-dependent bioactivation (Fitzsimmons, et al. (1995) Biochemistry 34:4276-4286). Moreover, the fatty acid side-chain of the antidepressant tianeptine is biotransformed by fatty acid β-oxidation (Fromenty, et al. (1989) Biochem. Pharmacol. 38:3743-3751). Further, 2-methyl fatty acids are substrates for β-oxidation (Mao, et al. (1995) Arch. Biochem. Biophys. 321:221-228). Accordingly, the antioxidant prodrugs of the present invention can be activated by one or more enzymes of fatty acid β-oxidation including, but not limited to, isovaleryl-CoA dehydrogenase, acyl-CoA transferase, thiolase, acyl-CoA dehydrogenase, enoyl-CoA hydratase, etc.

A mitochondria-targeted antioxidant prodrug of the present invention finds application in methods of decreasing the degree of mitochondrial dysfunction resulting from changes in the mitochondrial redox environment and preventing or treating a disease associated with mitochondrial dysfunction. As such, antioxidant prodrugs disclosed herein can be used alone or in admixture with a pharmaceutically acceptable carrier at an appropriate dose. Such pharmaceutical compositions can be prepared by methods and contain carriers which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. A pharmaceutically acceptable carrier or vehicle, e.g., a liquid or solid filler, diluent, excipient, or solvent encapsulating material, is involved in carrying or transporting the antioxidant prodrug from one organ, or portion of the body, to another organ, or portion of the body. Each carrier must be acceptable in the sense of being compatible with the other ingredients of the formulation and not injurious to the patient.

Examples of materials which can serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and other antioxidants can also be present in the compositions.

The compositions of the present invention can be administered parenterally (for example, by intravenous, intraperitoneal, subcutaneous or intramuscular injection), topically (including buccal and sublingual), orally, intranasally, intravaginally, or rectally according to standard medical practices.

The selected dosage level will depend upon a variety of factors including the activity of the particular antioxidant, the route of administration, the time of administration, the rate of excretion or metabolism of the particular compound being employed, the duration of the treatment, other drugs and/or materials used in combination with the particular antioxidant employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of an antioxidant and increase or decrease the levels as required in order to achieve the desired therapeutic effect. This is considered to be within the skill of the artisan and one can review the existing literature on a specific compound or similar compounds to determine optimal dosing.

Traditionally, mitochondrial diseases have focused on mitochondrial respiratory-chain diseases associated with mutations of mitochondrial DNA (DiMauro and Schon (2003) N. Engl. J. Med. 348:2656-2668). For instance, a maternal inheritance of a point mutation in the complex I gene (ND4) causes Leber's hereditary optic neuropathy (LHON) (Wallace, et al. (1988) Science 242:1427-1430). The diseases that result from familial mitochondrial DNA deletions and mutations are not as common as those that result from nuclear DNA defects. This may be because mitochondria contain several copies of their genome; hence, continuous fusion of mitochondria mixes the modified genes with the normal genes so that deleterious effects are reduced. Moreover, the continuous fission of mitochondria increases the likelihood that modified mitochondrial genes are removed by autophagy (Scheffler (1999) Mitochondria, Wiley-Liss, New York).

The realization that acute and chronic stresses to the cells lead to structural and functional impairments of mitochondria has redefined the role of mitochondria in disease etiology. Mitochondrial dysfunction resulting from changes in the mitochondrial redox environment triggers signaling cascades for necrosis and apoptosis of cells and results in organ failure and diseases. The list of diseases associated with changes in the mitochondrial redox environment includes, among others, cancer, heart failure, diabetes, obesity, stroke, neurodegenerative diseases, atherosclerosis, sepsis, and aging. As a result of changes in the mitochondrial redox environment these diseases all share the common features of disturbances of mitochondrial Ca²⁺, ATP, or ROS metabolism (Brookes, et al. (2004) Am. J. Physiol. 287:C817-C8330). For example, cancer cells show noticeable variation in their metabolic regulation and mitochondrial morphology and physiology compared with normal cells (Decaudin, et al. (1998) Int. J. Oncol. 12:141-152; Modica-Napolitano and Singh (2002) Expert Rev. Mol. Med. 2002:1-1-9). Antioxidants have been used to increase efficacy of anticancer therapeutic agents by reducing their adverse effects on normal cells (Lamson and Brignall (1999) Altern. Med. Rev. 4:304-329). Similarly, mitochondrial dysfunction contributes to the progression of neurogenerative diseases, e.g., Parkinson's disease and stroke (Mattson (2003) Neuromol. Med. 3:65-94; Stavrovskaya and Kristal (2005) Free Radic. Biol. Med. 38:687-697). Myocardial ischemia-reperfusion injury also results in mitochondrial Ca²⁺ overload that subsequently leads to uncontrollable ROS generation and opening of mitochondrial permeability transition pore (Brookes, et al. (2004) supra). Therefore, a mitochondria-targeted antioxidant prodrug of the present invention can be used for decreasing mitochondrial dysfunction by minimizing mitochondrial Ca²⁺ overload, decreasing mitochondrial ROS accumulation, or improving mitochondrial energy production. In this regard, prevention and treatment of the above-mentioned diseases is achieved.

Accordingly, the present invention is also a method of using the instant mitochondria-targeted antioxidant prodrug for decreasing the degree of mitochondrial dysfunction resulting from changes in the mitochondrial redox environment. This method of the invention involves contacting a cell with an effective amount of a mitochondria-targeted antioxidant prodrug such that upon activation by an enzyme of mitochondrial fatty acid beta-oxidation, the antioxidant is released from its prodrug form and decreases mitochondrial dysfunction. Depending on the action of the antioxidant, effectiveness of a mitochondria-targeted antioxidant prodrug can be monitored using any established method. For example, protection of mitochondria from oxidative damage and apoptosis is measured by determining lipid peroxidation (thiobarbituric acid reactive species), cytochrome c release, caspase-3 activation, DNA fragmentation, inactivation of complex I and aconitase, expression of transferrin receptor, mitochondrial iron uptake, and mitochondrial membrane potential. Moreover, uptake can be monitored using a nitrobenzofurazan derivative as disclosed herein.

As will be readily appreciated by one of skill in the art, the antioxidative activities of the instant prodrugs may also exhibit some antioxidative activities in the cytoplasm in the prodrug form, or alternatively, once activated leave the mitochondria and exert activity in the cytoplasm. Thus, while antioxidative actions primarily occur in the mitochondria, antioxidative activity is contemplated within the cellular domain from the plasmalemma through the cytoplasm, to golgi, to endoplasmic reticulum, to the mitochondria.

As indicated supra, the instant antioxidant prodrugs are also useful in the prevention and treatment of diseases or conditions associated with mitochondrial dysfunction resulting from changes in the mitochondrial redox environment. Prevention or treatment is achieved by administering to a subject an effective amount of a pharmaceutical composition of the present invention such that at least one sign or symptom of the disease or condition is ameliorated, delayed or inhibited. The amount administered can be dependent upon the disease to be treated, antioxidant being employed, and the pharmacokinetics and pharmacodynamics of the drug in the subject being treated.

Efficacy for the prevention and treatment of diseases or conditions associated with mitochondrial dysfunction can be monitored in a variety of well-established animal model systems for the diseases and conditions disclosed herein. For example, cardiac ischemia-reperfusion injury, which is associated with mitochondrial oxidative damage, can be investigated in a rat model (Adlam, et al. (2005) FASEB J. 19:1088-1095), wherein rats are given the antioxidant prodrug for a specified amount of time and observed for treatment-related effects on behavior or gross pathology. At the end of the treatment period, the hearts are removed and perfused in a Langendorff apparatus, which allows assessment of ventricular contractile function and left ventricular diastolic pressure (LVDP). After equilibration, the hearts are subjected to global zero-flow ischemia followed by normoxic reperfusion. LVDP, left ventricular pressure against time (dP/dt max), coronary blood flow, and heart rate are determined to assess efficacy.

The invention is described in greater detail by the following non-limiting examples.

### Example 1

### Fatty Acid β-Oxidation Activation of Phenol- and Pyridinol-Based Antioxidant Prodrugs

4-Thia- and 4-oxaalkanoic acids are biotransformed to alkanols and alkanethiols, respectively (Lau, et al. (1988) Biochemistry 27:5089-5095). Biotransformation of a generic 4-oxaalkanoate is presented in Scheme 1.

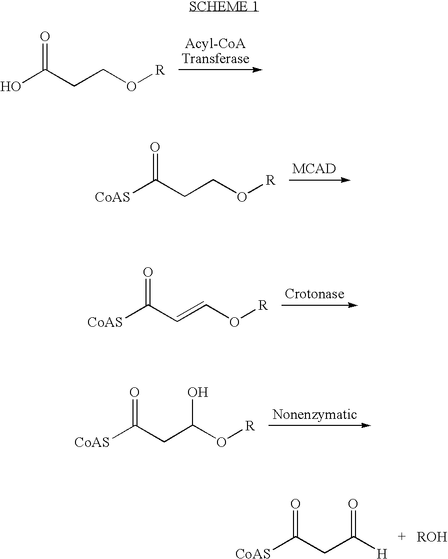

This reaction is catalyzed by the enzymes of fatty acid β-oxidation, which are localized in mitochondria. Using this catalytic activity, a variety of ROH or RSH groups can be delivered exclusively to mitochondria.

For example, phenolic antioxidants and 6-amino-3-pyridinols targeted to the mitochondria can be produced. Chain-breaking antioxidants, such as 2,6-dimethyl-4-methoxyphenol and α-tocopherol, inhibit lipid peroxidation.

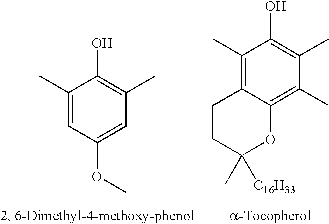

This group of antioxidants inhibits peroxidation by transferring their phenolic H atoms to the propagating radicals at a rate faster than that of chain propagation. The properties of a range of phenolic antioxidants have been determined by computational analysis (Wright, et al. (1997) J. Am. Chem. Soc. 119:4245:4252).

Moreover, a series of 6-amino-3-pyridinols, e.g., 1 and 2, has been found to be more effective than other phenolic antioxidants (Wijtmans, et al. (2003) Angew. Chem. Int. Ed. Engl. 42:4370-4373).

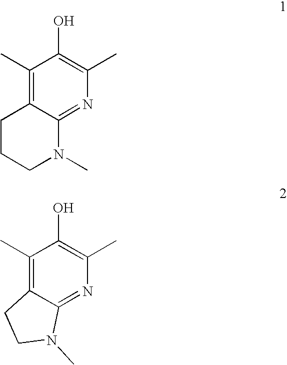

Pyridinol ethers 3 and 4 are the corresponding 4-oxabutanoic acid-based prodrugs of pyridinols 1 and 2.

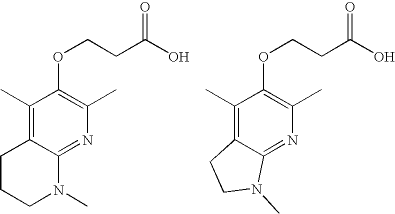

The synthesis of such antioxidants is achieved by the method disclosed in U.S. Pat. No. 6,770,672, incorporated herein by reference in its entirety, which discloses the synthesis of tocopherols, tocotrienols, and the like. Using this method, the chain lengthened analog, 3-(2,2,5,7,8-pentamethylchroman-6-yl)pentanoic acid, was synthesized. This prodrug is activated by fatty acid beta-oxidation conversion of the pentanoic acid analog to the propanoic acid followed by one more cycle of beta-oxidation, affording 2,2,5,7,8-pentamethylchroman-6-ol, the desired antioxidant.

### Example 2

### Fatty Acid β-Oxidation Activation of 4-Mercaptoimidzale (Ovothiol)-Based Antioxidant Prodrugs

Ovothiols are natural products found in sea urchins. The fertilization of sea urchin eggs is accompanied by the release of hydrogen peroxide, which results in the formation of a protective envelope by crosslinking tyrosine residues. The sea urchin egg is protected from the deleterious effects of hydrogen peroxide by the concomitant release of redox active 4-mercaptohistidines, termed ovothiols (Shapiro (1991) Science 252:533-536; Turner, et al. (1986) J. Biol. Chem. 261:13056-1 3063).

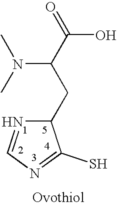

The redox activity of ovothiols is attributable to their ability to scavenge free radicals and their ability to function as nonenzymatic peroxidases (Shapiro & Hopkins (1991) Adv. Enzymol. 64, 291-3 16). The chemical property of ovothiols that contributes to their antioxidant potential, and distinguishes them from glutathione, is the pKₐ of the thiol group. The pKₐ of the thiol group of 1,5-dimethyl-4-mercaptoimidazole is 2.3 (Holler & Hopkins (1988) J. Am. Chem. Soc. 110:4837-4838); hence, at pH 7.0, mercaptoimidazoles exist almost completely (99.9%) in the imidazolium thiolate form. In contrast, the pKₐ, of the thiol group of glutathione is 8.6; at pH 7.0, glutathione exists largely (>90%) as the thiol. Accordingly, ovothiols react with iodoacetamide nine-times faster than glutathione. Ovothiols are effective scavengers of free radicals (Holler & Hopkins (1990) Biochemistry 29:1953-1961). 1,5-Dimethyl-4-mercaptoimidazole is much more effective than glutathione in reducing Fremy's salt and Banfield's radical. Hence, ovothiols are kinetically superior to glutathione in reducing free radicals, and this kinetic superiority is attributable to the resonance stabilization by the imidazole group.

Ovothiols may also serve as two-electron reductants. Ovothiols react more rapidly than glutathione with hydrogen peroxide (Turner, et al. (1988) Science 242:939-941); the second-order rate constants for the reaction of ovothiols and glutathione with hydrogen peroxide are 2.0 s⁻¹ M⁻¹ and 0.43 s⁻¹ M⁻¹, respectively.

The preparation and analysis of a panel of ovothiol-derived 4-mercaptoimidazoles has been described (Zoete, et al. (1997) J. Chem. Soc., Perkin Trans. I, 2983-2988). These studies showed that compounds bearing an electron-withdrawing group (3-ClC₆H₄, 2-ClC₆H₄, 2-CF₃C₆H₄, 3-CF₃C₆CH₄, CF₃) at C-2 had the highest radical scavenging ability. Subsequent studies have shown that these compounds are powerful scavengers of HOCl and are more potent than N-acetylcysteine (Zoete, et al. (2000) Free Radic. Res. 32:515-524). The radical-scavenging mechanism of 4-mercaptoimidazoles was studied by QSAR, cyclic voltammetry, ESR, and NMR spectroscopy, and a significant correlation was found between the DPPH scavenging abilities of the 4-mercaptoimidazoles and thermodynamic parameters (Zoete, et al. (2000) Free Radic. Res. 32:525-533).

Accordingly, mitochondria-targeted ovothiol-based prodrugs are disclosed herein for use in specifically scavenging mitochondrial ROS. A variety of 4-mercaptoimidazole antioxidants are well-known in the art (Spaltenstein, et al. (1987) J. Org. Chem. 52, 2977-2979; U.S. Pat. No. 4,898,878) and can be employed as reagents for the synthesis of mitochondria-targeted ovothiol-based prodrugs. For example, 1,5-dimethyl-4-mercaptoimidazole 6 with either ethyl acrylate 7 (Scheme 3) or ethyl 3-bromopropanoate 8 (Scheme 4) will, after hydrolysis of the ester (ethyl 3-(1,5-dimethyl-1H-imidazol-4-ythio)propanoate 9), give the desired sulfide, 3-(1,5-dimethyl-1H-imidazol-4-ythio)propanoic acid 10.

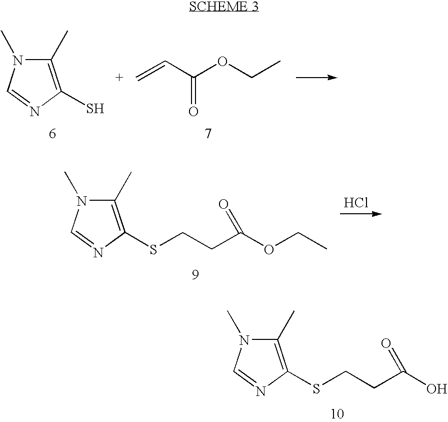

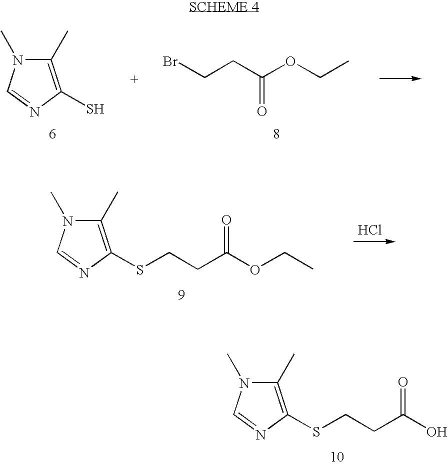

### Example 3

### Fatty Acid β-oxidation Activation of 3-Aminoxypropanoic Acid-Based Antioxidants

Although hydroxylamine (HONH₂) has long been known to possess anticancer activity and to delay senescence in mice (Harman (1961) J. Gerontol. A Biol. Sci. Med. Sci. 16:247-254), the observation of the retardation of senescence and the radioprotective effects of N-alkylhydroxylamines has only recently been reported. The spin-trapping compound α-phenyl-N-tert-butylnitrone (PBN) exerts well-described antiaging effects in vivo, delays senescence of normal human lung fibroblasts (IMR90), and has radioprotective effects in vivo (Kotake (1999) Antioxid. Redox Signal. 1:481-499; Lee & Park (2003) Cancer Res. 63:6885-6893). The observations that PBN decomposes to give N-tert-butylhydroxylamine and that old solutions of PBN are more effective than freshly prepared solutions of PBN in delaying senescence in IMR90 cells lead to the investigation of the antioxidant properties of N-tert-butylhydroxylamines and related N-substituted hydroxylamines (Chamulitrat, et al. (1995) Free Radic. Res. 23:1-14; Atamna, et al. (2000) J. Biol. Chem. 275:6741-6748).

N-tert-butylhydroxylamine, N-methylhydroxylamine, and N-benzylhydroxylamine, but not the O-methyl, O-tert-butyl, and O-benzyl analogs, delay senescence in IMR90 cells at concentrations as low as 10 μM (Atamna, et al. (2000) supra); in addition, N-substituted hydroxylamines delay senescence-dependent changes in mitochondria, prevent the age-associated decline in mitochondrial aconitase activity, block hydrogen peroxide-induced senescence, decrease the formation of ROS and oxidant-induced DNA damage, increase the glutathione/glutathione disulfide ratio, and inhibit the reduction of cytochrome c by superoxide. These findings show that oxidative phenomena contribute to cellular senescence and that N-substituted hydroxylamines effectively retard these changes. Subsequent studies confirmed these observations and further showed that N-tert-butylhydroxylamine 11 is oxidized to N-tert-butylhydronitroxide 12 and thence to 2-methyl-2-nitrosopropane 13, which are reduced to N-tert-butylhydroxylamine by mitochondrial NADH (Atamna, et al. (2001) FASEB J. 15:2196-2204) (Scheme 5).

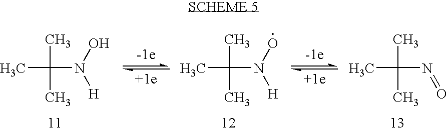

The radioprotective effects of N-tert-butylhydroxylamine have been studied in U937 cells and in mice (Lee, et al. (2004) Carcinogenesis 25:1435-1442). Ionizing radiation-induced cytotoxicity, cellular oxidative damage, and mitochondrial damage were all decreased by N-tert-butylhydroxylamine. Feeding N-tert-butylhydroxylamine (5 mg/kg daily for two weeks) to mice decreased the radiation sensitivity of animals subjected to 8 Gy of whole-body irradiation, and no compound-associated toxicity was observed. Although N-substituted hydroxyamines appear to induce little or no toxicity at the doses studied, hydroxylamine and O-substituted hydroxylamines are hematotoxic (Evelo, et al. (1998) Blood Cells Mol. Dis. 24:280-295)).

A series of 3-aminoxypropanoic acids has been synthesized as bioisosteres of antiinflammatory arylacetic acids, e.g., dichlofenac (Macchia, et al. (1990) J. Med. Chem. 33:1423-1430; Macchia, et al. (1995) Farmaco 50:83-90; EP 0 175 304). Several of these compounds show significant antiinflammatory activity in the carrageenan-induced paw edema test and some show platelet anti-aggregating activity. These compounds may, as the original experimental design proposed, serve as bioisosteres of antiinflammatory arylacetic acids; however, it is believed that these compounds may also undergo fatty acid β-oxidation-dependent metabolism to N-substituted hydroxylamines.

Accordingly, N-alkyl 3-aminoxypropanoic acids 14 are disclosed herein as antioxidant prodrugs, which upon fatty acid β-oxidation, release N-alkylhydroxylamines 15 (Scheme 6). Likewise, N-benzyl-3-aminoxypropanoic acid prodrugs are disclosed herein because these compounds also exhibit antioxidant potential and the aromatic ring imparts useful UV absorption.

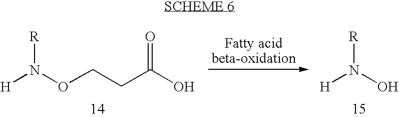

N-tert-butyl- and N-benzyl-3-aminoxypropanoic acid can be synthesized as shown in Scheme 7. N-Substituted 3-aminoxypropanoic acids are accessible by the Michael addition of the N-substituted hydroxylamines 15 to acrylamide 16 to give N-substituted 3-aminoxypropanenitriles 17 (Sayigh, et al. (1964) J. Org. Chem. 29:2042-2043); hydrolysis of the intermediate nitrile gives the N-substituted 3-aminoxypropanoic acids 18.

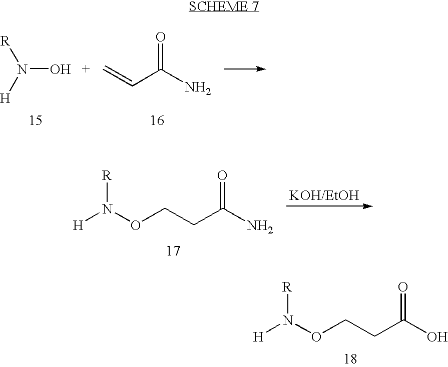

The required N-tert-butylhydroxylamine hydrochloride and benzylhydroxylamine hydrochloride are commercially available. The addition of N-alkylhydroxylamines to α,β-unsaturated esters can be considered as an alternative route to the desired 3-aminoxypropanoic acids, but reaction of hydroxylamines with α,β-unsaturated esters gives isoxazolones as products (Fountain, et al. (1975) Tetrahedron Lett. 3027-3030).

### Example 4

### Thiol-Based Antioxidant Prodrugs

4-Thiaalkanoates undergo mitochondrial β-oxidation to form butanethiol (from 4-thiaoctanoic acid; Lau, et al. (1988) Biochemistry 27:5089-5095). Haloalkene-derived 4-thiaalkanoates are potent mitochondrial poisons and also undergo β-oxidation-dependent bioactivation (Fitzsimmons & Anders (1993) Chem. Res. Toxicol. 6:662-668; Fitzsimmons, et al. (1995 supra). Hence, it is established that the mitochondrial β-oxidation can be used to deliver thiols to the mitochondria. 1,4-Dithiothreitol protects cells from S-(1,2-dichlorovinyl)-L-cysteine-induced expression of hsp70 (Chen, et al. (1992) J. Biol. Chem. 267:24322-24327), indicating a role for thiols in cytoprotection.

Accordingly, 2-(1,3-dithiolan-2-yl)acetic acid 19 (Scheme 8) and 2-(1,3-dithian-2-yl)acetic acid 20 (Scheme 9) are useful prodrug forms of cytoprotective dithiols. Both compounds can be considered to be analogs of isovaleric acid (3-methylbutanoic acid). Isovaleryl-CoA dehydrogenase catalyzes the conversion of isovaleryl-CoA to 3-methylcrotonyl-CoA (Finocchiaro, et al. (1987) J. Biol. Chem. 262:7982-2989), which is converted by enoyl-CoA hydratase to 3-hydroxyvaleryl-CoA. As such, 2-(1,3-dithiolan-2-yl)acetic acid 19 would yield ethane-1,2-dithiol 21 as a terminal product. With 2-(1,3-dithian-2-yl)acetic acid 20 as the substrate, the terminal product would be propane-1,3-dithiol 22.

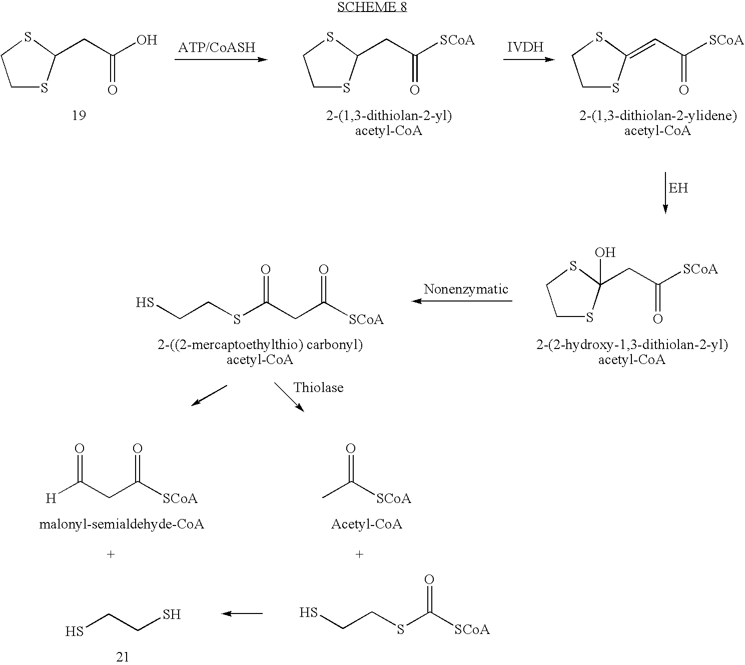

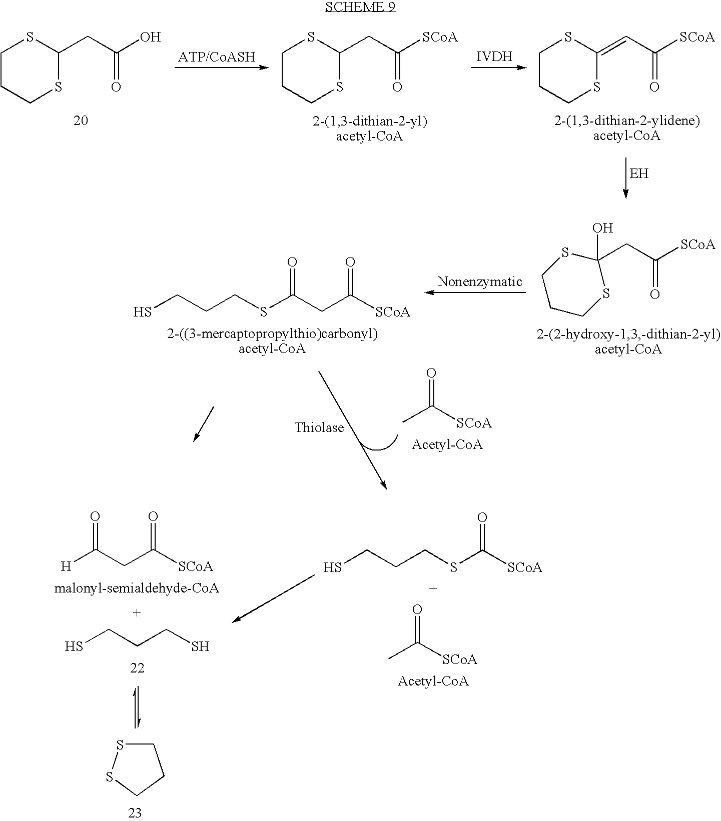

Unlike ethane-1,2-dithiol 21, propane-1,3-dithiol 22 can be oxidized (by analogy to 1,4-dithiothreitol) to 1,2-dithiolane 23. 2-((2-Mercaptoethylthio)carbonyl)acetyl-CoA and 2-((3-mercaptopropylthio)carbonyl)acetyl-CoA can be readily hydrolyzed or can serve as a substrate for 3-oxoacyl-CoA thiolase.

Synthesis of both (1,3-dithian-2-yl)acetic acid 20 and (1,3-dithiolan-2-yl)acetic acid 19 is shown in Scheme 10. The syntheses start with commercially available (Aldrich) methyl 3,3-dimethoxypropionate 24. Cleavage of the dimethylacetal and reaction of the resulting aldehyde with ethane-1,2-dithiol or propane-1,3-dithiol would give methyl 2-(1,3-dithioian-2-yl)acetate and methyl 2-(1,3-dithian-2-yl)acetate, respectively. Hydrolysis of the esters would give 2-(1,3-dithiolan-2-yl)acetic acid 19 and 2-(1,3-dithian-2-yl)acetic acid 20. An alternative route to (1,3-dithiolan-2-yl)acetic acid has been reported (Jones & Kropp (1974) Synthetic Commun. 4:331-334).

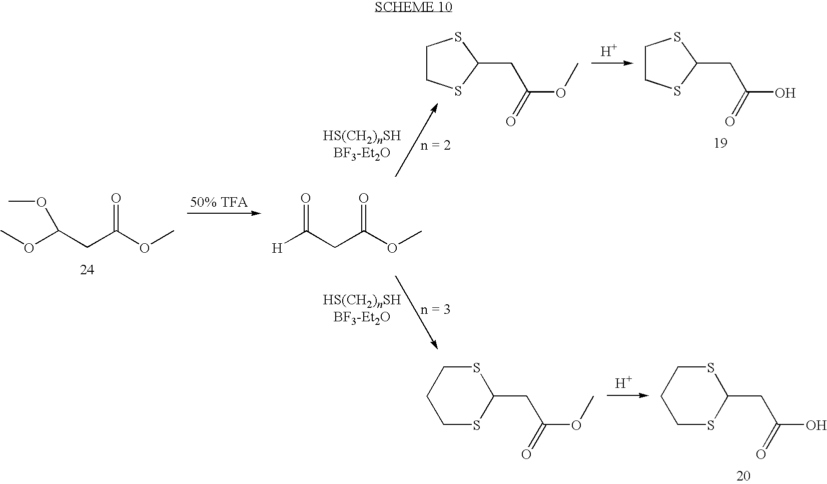

### Example 5

### Monitoring Mitochondrial Uptake

Renal organic cation transport has been analyzed using [2-(4-Nitro-2,1,3-benzoxadiazol-7-yl)aminoethyl] trimethylammonium 39 (Bednarczyk, et al. (2000) Pflügers Arch. 440:184-192). NBD-TMA is fluorescent, which allows measurement of the activity of transport systems in real-time. The fluorescent properties of nitrobenzofurazan (NBF) is useful for studying mitochondrial uptake of compounds disclosed herein in real-time. Although NBD-TMA itself is transported into mitochondria (it is similar to choline esters), modification of the NBF nucleus allows the study of transport of a range of compounds.

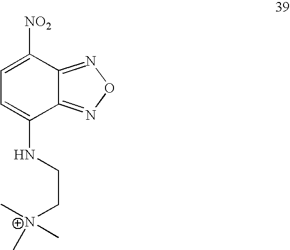

The choline ester of 7-carboxy-4-nitrobenzofurazan 40 allows investigation of uptake. Comparison of triphenylphosphonium-NBD 41 and trimethylammonium-NBD 42 allows comparison of the uptake of a hydrophilic and hydrophobic charged compound.

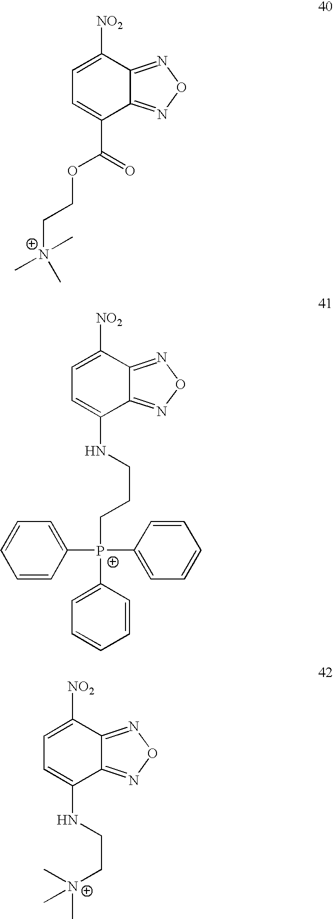

Several compounds can be prepared from the commercially available 4-chloro-7-nitrobenzofurazan. 4-Carboxy-7-nitrobenzofurazan (CAS 32863-22-2) has also been prepared (Dal Mone, et al. (1970) Annal. Chim. 60:801-814) and can be used for prepare the choline ester 40. 4-Hydroxy-7-nitrobenzofurazan (CAS 22250-54-0) has been prepared (Uchiyama, et al. (1998) J. Chem. Soc., Perkin Trans. 2, 2165-2174) and can also be used to prepare compounds. The fluorescent properties of a range of 4-substituted 7-nitrobenzofurazans has been investigated (Uchiyama, et al. (1998) supra).

What is claimed is:

1. A mitochondria-targeted antioxidant prodrug comprising a selected, fatty acid-modified antioxidant which is activated by an enzyme of mitochondrial fatty acid beta-oxidation.

2. A method for producing a mitochondria-targeted antioxidant prodrug comprising modifying a selected antioxidant to a fatty acid to produce a mitochondria-targeted antioxidant prodrug which is activated by an enzyme of mitochondrial fatty acid beta-oxidation.

3. A pharmaceutical composition comprising the mitochondria-targeted antioxidant prodrug of claim 1 in admixture with a pharmaceutically acceptable carrier.

4. A method for decreasing the degree of mitochondrial dysfunction resulting from changes in the mitochondrial redox environment comprising contacting a cell with an effective amount of a mitochondria-targeted antioxidant prodrug of claim 1.

5. A method for preventing or treating a disease associated with mitochondrial dysfunction resulting from changes in the mitochondrial redox environment comprising administering to a subject an effective amount of a pharmaceutical composition of claim 3.
